(19) United States
(12) Patent Application Publication
(10) Pub. No.: US 2021/0299319 A1
(43) Pub. Date: Sep. 30, 2021

(54) PATHOGENIC STERILIZATION WITH DEVICES EMITTING STERILIZING DOSES OF RADIATION

(71) Applicants: Armaan Gupta, Voorhees, NJ (US); Gia Gupta, Voorhees, NJ (US); Karina Gupta, Voorhees, NJ (US); Sanjay Gupta, Voorhees, NJ (US)

(72) Inventors: Armaan Gupta, Voorhees, NJ (US); Gia Gupta, Voorhees, NJ (US); Karina Gupta, Voorhees, NJ (US); Sanjay Gupta, Voorhees, NJ (US)

(21) Appl. No.: 17/217,162

(22) Filed: Mar. 30, 2021

Related U.S. Application Data
(60) Provisional application No. 63/002,243, filed on Mar. 30, 2020.
(60) Provisional application No. 63/036,276, filed on Jun. 8, 2020.

(51) Int. Cl. A61L 9/20; A61L 2/00; A61L 2/10; A61L 2/26; A61M 16/10; A61M 16/06

(52) U.S. Cl. CPC A61L 9/20; A61L 2/0047; A61L 2/10; A61L 2/26; A61L 2202/23; A61M 16/06; A61L 2202/11; A61L 2209/12; A61M 16/10

(57) ABSTRACT

The present disclosure is related to objects, apparatuses, devices, and methods of utilizing UV-C radiation to sterilize pathogenic contamination of materials such as air and bodily tissues.

### CROSS REFERENCE TO RELATED APPLICATIONS

The present application claims priority under 35 U.S.C. § 119 to U.S. App. No. 63/002,243, filed Mar. 30, 2020, and U.S. App. No. 63/036,276, filed Jun. 8, 2020, each of which are hereby incorporated by reference in their entirety.

### FIELD OF DISCLOSURE

The present disclosure is related apparatuses, devices, and methods intended to be used to administer sterilizing doses of radiation. In particular, the sterilizing dose of radiation may be used in air to be inhaled, or the mucosa of a user, or a surface to sanitize these of pathogenic materials.

### BACKGROUND

A common medium for certain pathogenic agents, such as bacteria, viruses and other organisms, to spread is on surfaces. The virus may be transmitted to the surface, and upon human contact spread to a host. Upon contact with the host, the pathogenic agent may infect the host and/or the host may carry the pathogenic material thus increasing infection density among a populace.

Certain electromagnetic radiation is capable of sterilizing surfaces therefore reducing the number of pathogenic agents on that surface. For example, some short-wavelength ultraviolet radiation is capable of killing or inactivating microorganisms by, for example, destroying nucleic acids and disrupting the RNA/DNA of the organisms and thus leaving the microorganism unable to perform vital cellular functions. An ideal UV disinfection model follows first order kinetics whereby the pathogenic microorganism density remaining after exposure to UV (N) is given by:

$$N = {N}_{0} {e}^{- kIt}$$

where Nₒ is the initial microorganism density, k is a rate constant proportional which relates to factors such as the likelihood of microorganism absorption and RNA/DNA damage, I is the intensity of radiation, and t is the time of exposure.

Electromagnetic based sterilization devices are often separate from the object and require repeated non-continuous use in order to decrease the number of pathogens such as bacteria or virions present in the contaminated material. Furthermore, these devices require direct line of sight exposure to the sterilizing radiation. Accordingly, the sterilization efficacy may be limited by surface exposure and times of exposure to the radiation.

Also, prior use of UV light in humans (internal and external use) has been focused on using UV-A and UV-B radiation. Light of these wavelengths has limited viricidal capacity and thus requires long time of exposure. At these long exposure times, the light is often harmful to body tissues such as the oral and nasal mucosa. Several studies suggest that UV-A and UV-B substantially penetrate body tissues thus resulting in increased harm to subjects administered these sterilizing doses.

It is therefore an object of this disclosure to provide objects, apparatuses, and methods of exposing materials to this sterilizing radiation which decrease viral loads therein.

### SUMMARY

In accordance with the foregoing objectives and others, the present disclosure provides materials with electromagnetic radiation dispersed within the surface capable of sterilizing surface of the material when the radiation is dispersed through the air/material interface. The objects may afford increased exposure time and/or intensity as compared to sterilization techniques involving bombarding the surface with radiation from the air to the surface. Additionally, by providing increased or constant exposure, the microorganism density on these surfaces may not increase to a level high enough to allow for probable transmission or infection to objects which come in contact with the surface (e.g., the number of one or more microorganisms transmitted to a host is less than 50% or less than 25% or less than 10% or less than 5% or less than 1% as compared to an otherwise identical object without the sterilizing radiation distributed therethrough).

These self-sterilizing objects may comprise a sterilizing radiation source capable of emitting a sterilizing dose to the surface of the object; wherein the sterilizing radiation source may be embedded within or otherwise positioned to pass the sterilizing radiation through a transparent or translucent body of the object. In these configurations, the sterilizing radiation may be dispersed and/or pass through the transparent or translucent body of the object such that the light is emitted from the surface to the air at the air/surface interface.

The sterilizing radiation may be any electromagnetic radiation capable of sterilizing the surface and decreasing the pathogenic agents. For example, the sterilizing radiation may be ultraviolet radiation (e.g., UV-C radiation which may have a wavelength of less than 300 nm or a wavelength of from 200 to 300 nm), optical radiation, infrared radiation, or combinations thereof. The power intensity of radiation at the surface may be (which correlates with the sterilizing dose), for example, less than 100 mJ/cm² less than 60 mJ/cm² or less than 50 mJ/cm² or less than 40 mJ/cm² or less than 30 mJ/cm² or less than 20 mJ/cm² or less than 10 mJ/cm² or less than 5 mJ/cm² or from 1 mJ/cm² to 60 mJ/cm² or from 1 mJ/cm² to 25 mJ/cm² or from 1 mJ/cm² to 10 mJ/cm².

The object comprising the transparent or translucent material may be any surface typically touched by members of a populace. For example, in some embodiments the object may be any frequently touched surface such as a fixture on a door or countertop, a doorbell, a door, a doorknob, door handle, toilet, flush handle of a toilet, hair net, utility handle such as a shopping cart handle, implements for holding, transferring, or storing food such as tongs or reusable food storage containers, face shields, facemasks such as surgical face masks, a hand covering device such as hand gloves. In some embodiments, the object may be eye glasses, a cannula (e.g., nasal cannula), jewelry such as an earring, nose ring, or stud, arm band, wound cover, bandages of the skin, medical implants such as those which may be left inside the body, a wound, or skin of a user. The medical implant may be in the form of a capsule, bulb, catheter, or electrode. Body implants and catheters may be modified to embed sterilizing electromagnetic radiation of the present disclosure. For example, catheters for insertion in blood vessels, heart, spinal cord, bladder, and the like may comprise the sterilizing radiation of the present disclosure. In some embodiments, the object may a micro implant for insertion in body cavities, or to be placed, for example, orally, anally, aurally (ear canal), nasally and/or in the sinuses, and other body orifices such as the stomach, sinus, bladder and other portions of the body which may become infected. In some embodiments, the object may be skull cap, or a cap worn around the hair. In embodiments worn around the hair, the sterilizing surface may decrease incidence of dandruff as well.

In some embodiments, the surface comprises a coating at the air/surface interface. The coating may allow for diffraction of sterilizing radiation at the coating/surface interface resulting from different refractive indexes between the two materials and the sterilizing radiation is provided such that a sterilizing dose of radiation is provided at the air/coating interface. In some embodiments, the surface is uncoated. In various implementations, the surface does not include a coating of a UV active photocatalyst, such as a TiO₂ photocatalyst. In some embodiments, the surface includes a coating of a UV active photocatalyst, such as a TiO₂ photocatalyst.

The present disclosure also provides apparatuses, devices, and methods of use thereof which are capable of administering sterilizing radiation comprising UV-C radiation. For example, the sterilizing radiation may emit photons having one or more wavelengths from 10 nm to 400 nm. In certain embodiments, the sterilizing may comprise UV-C light, alone or in various combination of other light radiation. In various implementations, the sterilizing may comprise more than 50% or more than 60% or more than 70% or more than 80% or more than 90% or more than 95% or more than 99% UV-C radiation (e.g., light having a wavelength from 10 to 400 nm) as measured by intensity. In particular, these devices may sterilize air or certain tissues associated with inhalation such as the oral and nasal mucosa. By utilizing UV-C radiation, different modalities and treatment regimens may be realized as compared to sterilizations involving lower energy radiation (e.g., UV-A, UV-B). The sterilizing doses of the present disclosure may have increased pathogenic sterilization efficacy and allow for decreased exposure time for sterilization which is particularly relevant when sterilizing bodily tissues.

An apparatus for delivering sterilized air to a subject during inhalation of the present disclosure may comprise:
a) a solid enclosure having an internal chamber configured to allow air to pass through the internal chamber and be delivered to the subject;
b) optionally one or more delivery ports in fluid communication with the internal chamber; wherein air passes through the port for delivery to the subject for inhalation; and
c) a sterilizing radiation source capable of emitting:
   i) a sterilizing dose of radiation delivered to the air present in the internal chamber prior to delivery to the subject (e.g., the air flowing through the internal chamber during inhalation);
   ii) a sterilizing dose of radiation delivered to one or more of the oral cavity of the subject, the nasal cavity of the subject, and the air that has moved through the port; or
   iii) any combination of two or more of the foregoing.

In certain embodiments, the apparatus may comprise:
a) one or members dimensioned to be inserted into the nasal cavity; and
b) a sterilizing radiation source capable of emitting a sterilizing dose of radiation to the nasal cavity when the member is inserted in the nasal cavity.
   In some embodiments, the apparatus may be a cannula (e.g., nasal cannula), catheter, or penlight.

Objects are provided which may comprise a surface composed of a transparent or translucent material positioned at the air interface of the object and a sterilizing radiation source capable of emitting a sterilizing dose at the interface after transmission of the radiation through to the air interface. In some embodiments, the radiation source is embedded within the transparent or translucent material. In various implementations, the radiation source is proximal to the transparent or translucent material. In some embodiments, the radiation is delivered to the transparent or translucent material via an optical wave guide. In various aspects, the object is a doorknob, door handle, toilet, flush handle of a toilet, hair net, shopping cart handle, implement for holding, transferring or storing food, facemask. or a flat surface on the door or counter top. In some embodiments, the object is eye glasses, face shield, face mask, nasal cannula, nose ring or stud, arm band, wound cover, or bandage. In some embodiments, the object is a medical implant (e.g., in the form of a capsule, bulb, catheter, electrode).

### BRIEF DESCRIPTION OF FIGURES

FIG. 1A illustrates a face mask of the present disclosure on a user which sterilizes air prior to inhalation.

FIG. 1B illustrates a face mask of the present disclosure on a user which sterilizes air prior to inhalation utilizing a tube to increase the pathlength of air during sterilization.

FIG. 2A is a perspective view of an apparatus of the present disclosure.

FIG. 2B illustrate the insertion of the apparatus shown in FIG. 2A into the nasal cavity of a user to provide sterilizing radiation doses thereto.

FIG. 3 is a graph illustrating a spectrum of sterilizing radiation produced in an exemplary apparatus of the present disclosure. Wavelength is represented on the x-axis and intensity is represented on the y-axis. The dotted lines are the individual spectrum produced from the indicated emitter resulting in the bimodal spectral distribution.

FIG. 4 is a perspective view of an embodiment of the translucent or transparent surfaces described herein.

FIG. 5 is a perspective view of a cannula comprising the sanitizing transparent or translucent surfaces described herein.

FIG. 6 illustrates an apparatus for delivery of sterilized air according to the present disclosure.

FIG. 7A is a perspective view of an embodiment of the translucent or transparent surfaces described herein.

FIG. 7B is a cross sectional view of the embodiment depicted in FIG. 7A.

FIG. 8A is a cross sectional view of a door with a doorknob having the sterilizing transparent or translucent surfaces as described herein.

FIG. 8B is a cross sectional view of a door with a doorknob having the sterilizing transparent or translucent surfaces as described herein.

FIG. 9 is a perspective view of a door opening device on a door comprising the sanitizing transparent or translucent surfaces described herein.

### DETAILED DESCRIPTION

Detailed embodiments of the present disclosure are disclosed herein; however, it is to be understood that the disclosed embodiments are merely illustrative of the disclosure that may be embodied in various forms. In addition, each of the examples given in connection with the various embodiments of the disclosure is intended to be illustrative, and not restrictive.

All terms used herein are intended to have their ordinary meaning in the art unless otherwise provided. All concentrations are in terms of percentage by weight of the specified component relative to the entire weight of the topical composition, unless otherwise defined.

As used herein, “a” or “an” shall mean one or more. As used herein when used in conjunction with the word “comprising,” the words “a” or “an” mean one or more than one. As used herein “another” means at least a second or more.

As used herein, all ranges of numeric values include the endpoints and all possible values disclosed between the disclosed values. The exact values of all half-integral numeric values are also contemplated as specifically disclosed and as limits for all subsets of the disclosed range. For example, a range of from 0.1% to 3% specifically discloses a percentage of 0.1%, 1%, 1.5%, 2.0%, 2.5%, and 3%. Additionally, a range of 0.1 to 3% includes subsets of the original range including from 0.5% to 2.5%, from 1% to 3%, from 0.1% to 2.5%, etc. It will be understood that the sum of all % of individual components will not exceed 100%, unless otherwise indicated.

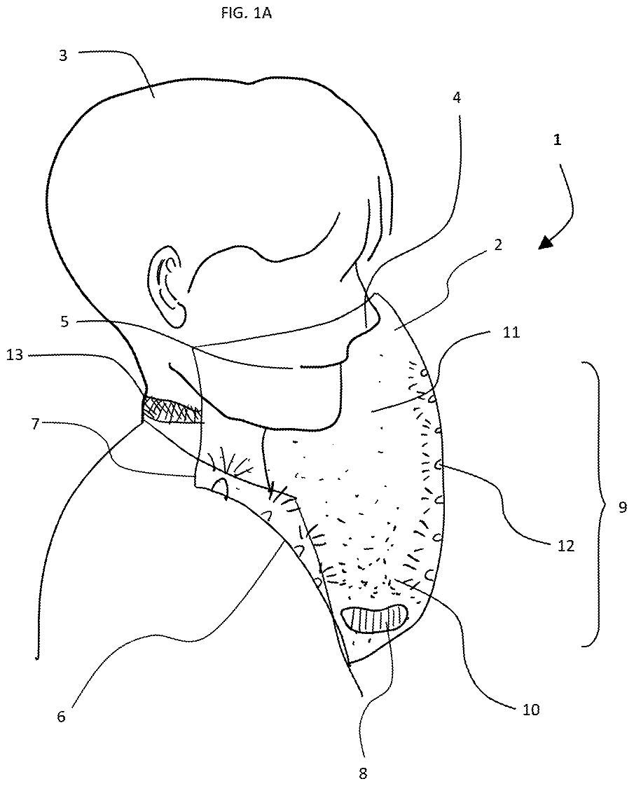

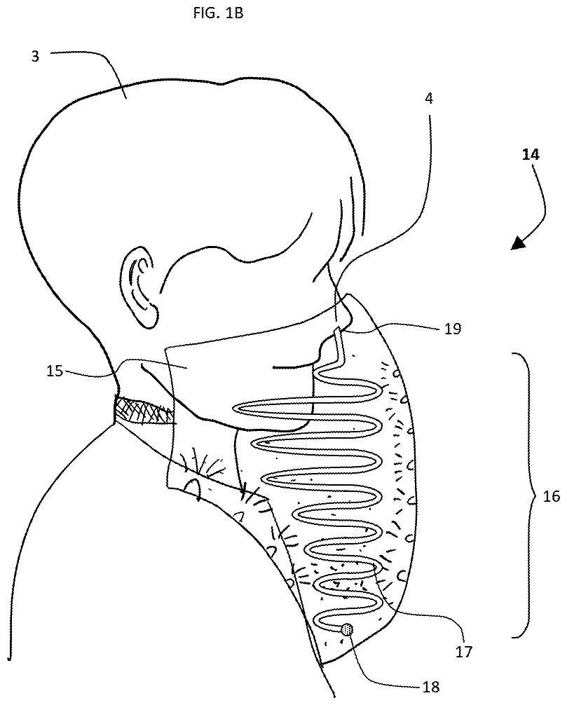

Referring now to FIG. 1, an apparatus 1 is illustrated configured as a face mask for user 2 which comprises a solid enclosure 2 which fits around the nose 4 and mouth 5 of user 2. Solid enclosure is dimensioned to be extend to the chest 6 and shoulders 7 of user 2 and for a large internal cavity which air may be sterilized prior to inhalation by the user. The internal cavity, or a portion thereof, may be formed between the barrier and the face of the user. In certain embodiments, the internal cavity has a volume of less than 2 L or less than 1.5 L or less than 1 L or less than 0.5 L. In certain embodiments, the volume may be changed by a user such as with a cinch or tie. As can be seen, configurations where the barrier extends below the user's chin allow for the creation of a large internal volume air which may be sterilized. Furthermore, by extending to chest 6 and/or shoulders 7, the face mask may be supported by the user's body and prevent movement away from the inhalation cavities. In some embodiments, the solid barrier may further cover a user's eyes. In some embodiments, the solid enclosure surrounds a user's head. Apparatus 1 is further secured and stabilized through band 13 which wraps around the back of the user's neck.

Air may enter into the internal volume of solid enclosure 2 through one or more air passageways 8 such as filters which are typically disposed away from the inhalation cavities (e.g., more than 5 cm away from the mouth and/or nose, more than 7 cm away from the mouth and/or nose, more than 0.10 cm away from the mouth and/or nose). Air may enter into the internal cavity through air passageway 8. In certain embodiments, the air enters through an air intake port which may regulate the flow of ambient air into the internal cavity. Apparatus 1 comprises a sterilizing radiation source 9 which emits sterilizing radiation to sterilize the ambient air moved into the internal cavity prior to inhalation. Adjusting the volume of the internal cavity or the position of the air passageway may alter the residence time of the ambient air in the internal cavity prior to inhalation. Accordingly, such adjustment may offer variations in sterilization capability of the device. For example, ambient air that has recently traveled into the internal cavity may have a larger number of pathogens therein at position 10, but as the ambient air flows through the internal cavity, towards position 11 the air may be increasingly sterilized (as represented by the change in density of pathogens between positions 10 and 11).

In a particular embodiment, the mask may be composed of plastic material capable of maintaining a modular form to form an internal cavity between the barrier and the face having a volume of from 100 cc to 1000 cc (e.g., 300-700 cc, 400-600 cc, 500 cc) of air at any given time. This particular range may be optimized to coincide with the average volume of air the subject inhales in each breath. Each breath may remove most of the sterilized air within the internal cavity. That removal will refill the internal cavity with ambient which, in turn, will be sterilized. Even if full seal between the mask and the user is not present these single breath embodiments may decrease the likelihood of nonsterilized air to be inhaled. In certain implementations, the mask may be supported at the upper chest level which may thus preventing slippage of the mask which would result in inhalation of non-sterilized air.

The masks of the present disclosure, and particularly the masks having an internal cavity volume of from 100 cc to 1000 cc (e.g., 300-700 cc, 400-600 cc, 500 cc) may comprise one or more air intake and/or exhaust ports which may control the flow rate of air into and/or out of the internal cavity. For example, ambient air may flow into the internal cavity with a set maximum flow rate of from 1 to 10 L/min (e.g., 3-8 L/min, 5-7 L/min). Suitable control may be achieved by, for example air intake ports comprising one or more valves such as needle valves, or one way valves, exhalation valves, output flow-control valves. The flow rate may be altered by the user to allow for control of the rate of air to come into the internal cavity which may provide a user control of the exertion required at each inhalation and the amount of pathogens that enter in each inhalation cycle. For example, users in a very polluted environment may have lower flow rates in order to allow for increased sterilization as less pathogens are pulled into the internal cavity. In less contaminated environments, a maximum flow of the air intake port rate may be increased thus making it easier to inhale, but still offering an appropriate sterilizing dose to the decreased density of airborne pathogens.

In some embodiments, masks of the present disclosure may further comprise a voice enhancer or voice modifier as disclosed in U.S. Pat. No. 4,683,588, which is hereby incorporated by reference in its entirety. The voice modifier may be put in the mask that may modulate the voice of the user, for example by altering the intensity and/or pitch of a user's voice while speaking with the mask on. The voice enhancer may, for example comprise an electronic component (e.g., the voice enhancer may comprise a microphone, speaker, individually or in combination) or a mechanical device such as those that that modulates sound based on vibration or air flow (e.g., mechanical larynx, electrolarynx). In certain embodiments, the face mask may comprise a voice modifier; wherein one or more of the components may be self-contained within the mask. For example, a microphone, electrically capturing the wearer's voice may be located proximal to the wearer's mouth. The microphone may be electrically connected to a voice signal modifying device which serves to alter the electrical signals transmitted by the microphone so that they, when connected to a speaker provides speech in a distorted manner from the wearer's actual voice if not affected by the device. The voice modifier may amplify in volume, muffle in volume, shift in frequency, mask high or low frequencies produce a monotone, computer-like voice from the wearer, individually or in any combination of two or more.

In various implementations, ambient air may be sterilized (e.g., air entering a mask) by flowing ambient air through one or more tubes having sterilizing radiation bombarding the flowing air as it passes through the tube to the user for inhalation. In certain implementations, increased tubes length may provide the extra contact time between the UV-C and air and therefore increased sterilization. In certain implementations, the tube may be used in the face masks of the present disclosure, for example providing air intake to the internal cavity, providing an exhaust port (e.g., proximal to the nasal orifices).

The sterilizing radiation source 9 may comprise a plurality of individual emitters 12 emitting radiation into the internal chamber to provide the sterilizing does to the air. Many individual emitters may be used to control the spectrum of the sterilizing radiation to produce a sterilizing dose appropriate to the medium being sterilized. For example, the sterilizing radiation source comprises a light emitting diode (LED) (e.g., an AlN LED, a BN LED, a diamond LED, a GaN LED, an AlGaN LED), a mercury lamp, or an excimer lamp (e.g., NeF excimer lamp, Ar₂ excimer lamp, Kr₂ excimer lamp, F₂ excimer lamp, ArBr excimer lamp, Xe₂ excimer lamp, ArCl excimer lamp, KrI excimer lamp, ArF excimer lamp, KrBr excimer lamp, KrCl excimer lamp, KrF excimer lamp, XeI excimer lamp, Cl₂ excimer lamp, XeBr excimer lamp, Br₂ excimer lamp, XeCl excimer lamp, I₂ excimer lamp, XeF excimer lamp), individually or in combinations of two or more thereof. Various emitters may be used to produce multimodal spectral distributions. For example, the sterilizing radiation spectrum may comprise one or more local spectral maximum wavelengths selected from 108 nm, 126 nm, 146 nm, 158 nm, 165 nm, 172 nm, 175 nm, 190 nm, 193 nm, 207 nm, 210 nm, 215 nm, 222 nm, 235 nm, 248 nm, 253 nm, 259 nm, 282 nm, 289 nm, 308 nm, 342 nm, 351 nm, or 365 nm. Each local spectrum having a spectral maximum wavelength may independently have a full-width half maximum of less than 50 nm or less than 40 nm or less than 20 nm or less than 10 nm or less than 5 nm or less than 1 nm.

In certain embodiments, the sterilizing radiation source may emit a sterilizing dose of radiation comprising UV-C light. For example, the sterilizing radiation may emit photons having one or more wavelengths from 10 nm to 400 nm. In certain embodiments, the sterilizing may comprise UV-C light, alone or in various combination of other light radiation. In various implementations, the sterilizing may comprise more than 50% or more than 60% or more than 70% or more than 80% or more than 90% or more than 95% or more than 99% UV-C radiation (e.g., light having a wavelength from 10 to 400 nm) as measured by intensity. In some embodiments, the spectrum is multimodal such as bimodal or trimodal. In various implementations, the sterilizing radiation source may emit radiation to be delivered to tissue (e.g., mucosa such as the nasal mucosa or the oral mucosa), and the sterilizing radiation source emits radiation comprising two spectral maximums having different penetration depths in the mucosa. The sterilizing radiation comprise may be surface sterilizing radiation which predominantly sterilizes the surface of the mucosa (e.g., more than 50% or more than 60% or more than 70% or more than 80% or more than 90% of the photons sterilize the surface of the mucosa). In particular, the surface sterilizing radiation may have a spectral maximum of less than 240 nm or less than 230 nm. In various implementations, the sterilizing radiation may be penetrating sterilizing radiation which predominantly sterilizes the mucosa beneath its surface (e.g., more than 50% or more than 60% or more than 70% or more than 80% or more than 90% of the photons do not sterilize the surface of the mucosa). For example, the surface sterilizing radiation may have a spectral maximum of more than 230 nm or more than 240 nm.

In certain implementations, the apparatus may be a face mask;

wherein the solid enclosure is dimensioned to be fit around the mouth and the nose of the subject during inhalation;
the solid enclosure is dimensioned to be positioned on the chest of the subject, the shoulders of the subject, or the chest and the shoulders of the subject during inhalation; and
the face mask is supported by the positioning on the chest of the subject, the shoulders of the subject, or the chest and the shoulders of the subject during inhalation. In various implementations, the apparatus may be a nasal cannula or catheter. In various implementations, the solid enclosure may comprise an air intake port and a tube comprising a distal end having an air delivery port dimensioned to be inserted into the nose of the subject and a distal end and air may flow through the distal end; wherein
said air intake port is in fluid communication with the distal and ambient air may flow through the air intake port, through the tube, and out the air delivery port;
said tube is transparent or translucent to the sterilizing radiation; and
said sterilizing dose of radiation is delivered to the ambient air flowing through the tube.
   Referring now to FIG. 1B, a face mask 14 is depicted being worn on a user 3 with nose 4. Facemask 14 has solid enclosure 15 and sterilizing radiation source 16, similar to those in FIG. 1A. Facemask 14 also comprises an air delivery implement which transports air between air intake port 18 and the nasal cavity of the user such as tube 17. The air delivery implement may augment the trajectory of the flowing air such that the air may have a path length longer than the distance between the air intake port and the user's nose. Such implements may allow for increased exposure to the sterilizing radiation produced from sterilizing radiation source 16 by increasing the pathlength of the air in the sterilizing region of the device. As can be seen, tube 17 has a substantially oscillating configuration along the pathlength to the nasal cavity. The air delivery implement may be composed of a flexible material to allow for such increased pathlengths. In some embodiments, the air delivery implement may have one or more portions removably attached to a surface of the solid enclosure. In some embodiments, the implement may have a length of less than 300 cm or less than 200 cm or less than 100 cm or less than 50 cm or less than 30 cm. In various implementations, the implement may have a length of from 10 cm to 500 cm or from 30 cm to 400 cm or from 30 cm to 200 cm.

In some embodiments, the object, apparatus, or device of the present disclosure may be a cannula (e.g., nasal cannula) or medical implants such as those which may be left inside the body, a wound, or skin of a user. The medical implant may be in the form of a capsule, bulb, catheter, or electrode. Body implants and catheters may be modified to embed sterilizing electromagnetic radiation of the present disclosure. For example, catheters for insertion in blood vessels, heart, spinal cord, bladder, and the like may comprise the sterilizing radiation of the present disclosure. In some embodiments, the object may a micro implant for insertion in body cavities, or to be placed, for example, orally, anally, aurally (ear canal), nasally and/or in the sinuses, and other body orifices such as the stomach, sinus, bladder and other portions of the body which may become infected. In some embodiments, the object may be skull cap, or a cap worn around the hair. In embodiments worn around the hair, the sterilizing surface may decrease incidence of dandruff as well.

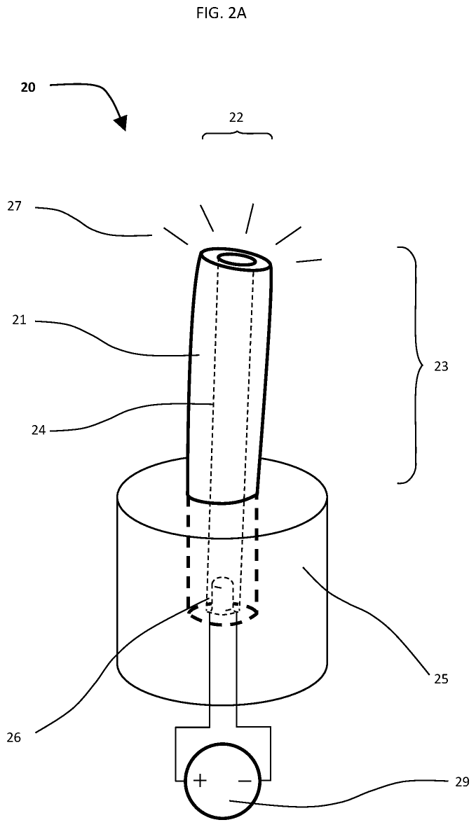

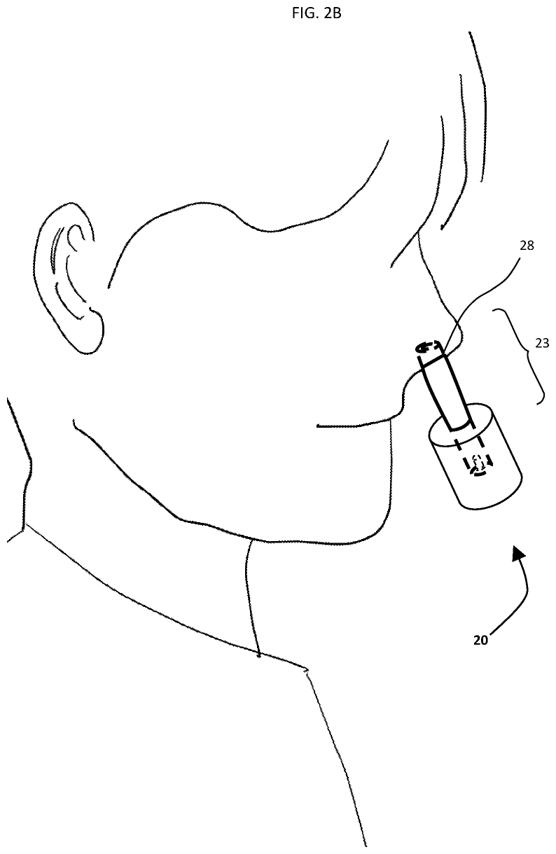

Referring now to FIGS. 2A and 2B, an apparatus 20 which may be used to sterilize a medium is illustrated. Apparatus 20 comprises a member 21 dimensioned to be inserted into a cavity such as a nasal cavity, an oral cavity, vaginal cavity, or rectal cavity of a user. The appropriate dimensions to identify for insertion include the width 22 and height 23. For example, the insertion member may be elongated and have a width of less than 5 cm or less than 4 cm or less than 3 cm or less than 2 cm or less than 1 cm or less than 0.1 cm or less than 10 mm. In certain implementations, the insertion member may have a height of less than 5 cm or less than 4 cm or less than 3 cm or less than 2 cm or less than 1 cm or less than 0.1 cm. In the embodiment depicted, insertion member 21 is elongated and comprises a generally cylindrical shape. The inhalation member 21 comprises an internal cavity 24 (internal surfaces denoted in dotted lines) which is inserted into a housing or holder 25. Within the housing or holder is a sterilizing radiation source 26 which emits radiation through the internal cavity 24 and out the distal end resulting in sterilizing radiation emission 27 which can be within an inhalation cavity of a user. As shown in FIG. 2B, apparatus 20 may be inserted into the nose of a user where only a portion 28 of the inhalation member is inserted into the nasal cavity along height 23. This portion 28 comprises the distal end wherein sterilizing light is emitted from the device. In some embodiments, the portion to be inserted in the cavity may have a maximum dimension of less than 5 cm or less than 4 cm or less than 3 cm or less than 2 cm or less than 1 cm or less than 0.1 cm or less than 10 mm. In some embodiments, the portion to be inserted in the cavity may be in the shape of a funnel or ear speculum which may provide for a wider spread of light inside the cavity. For example, the width of the insertion member (or portion to be inserted) may increase towards the distal end where sterilizing light is emitted. Sterilizing radiation source 27 is connected to a power source 29 in order to provide the appropriate power the to the sterilizing radiation source 27 which may comprise one or more individual emitters such as light emitting diodes, arc lamps, excimer lamps, used individually or in combinations thereof. For example, the power source may be an electrical outlet, a battery (such as a lithium ion battery), used individually or in combination. In various implementations the power source may be within housing 25.

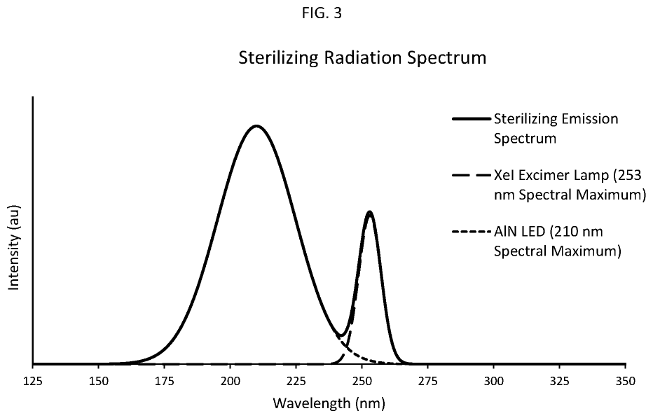

Apparatuses of the present disclosure such as those comprising internal cavity 24 may be easily integrated into medical devices such as cannula (e.g., nasal cannula, oral cannula) or catheters in order to provide a sterilizing dose of radiation to the bodily surface in contact therewith. For example, when integrated in a nasal cannula, the sterilizing radiation may sterilize the nasal mucosa while also delivering air (e.g., sterilized air) to the use for inhalation. In certain embodiments, the sterilizing may comprise UV-C light, alone or in various combination of other light radiation. In various implementations, the sterilizing may comprise more than 50% or more than 60% or more than 70% or more than 80% or more than 90% or more than 95% or more than 99% UV-C radiation (e.g., light having a wavelength from 10 to 400 nm) as measured by intensity. In some embodiments, the source of sterilizing radiation may comprise two or more individual emitters with different spectral wavelength maximums in order to sterilize different depths of the body surface which the sterilizing radiation interacts. Without wishing to be bound by theory, it is believed that light in the UV-C region having less energy (e.g., light having a wavelength above 230 nm) is able to penetrate deeper in certain substrates such as the nasal mucosa as compared to higher energy light (e.g., light having a wavelength below 230 nm). Combinations of various individual emitters of the present are able to produce a spectrum which will allow for sterilization in both of these regimes. For example, FIG. 3 illustrates the emission spectrum of a sterilizing emission source comprising one or more XeI lamps (having a maximum spectral wavelength of 253 and a full-width half maximum of 10 nm) and one or more AlN light emitting diodes (having a maximum spectral wavelength of 210 nm and a full-width half maximum of 35 nm). This bimodal spectrum allows for sterilizing radiation to sterilize the surface of the mucosa (from the light around the 210 nm maximum) and a depth within the mucosa (from the light around the 235 nm maximum).

Since lower wavelength (e.g., 200-240 nm such as 220 nm) may have less penetration to the mucosa, sterilizing radiation may have decreased tissue damage as compared to longer wavelength light. In various implementations, the sterilizing dose of radiation may comprise lower wavelength light alone or in combination with other radiations (e.g., UV-C radiation) in order to facilitate sterilization of pathogens inside and outside of the body. For example, the apparatuses of the present disclosure may be inserted into the nasal and other body cavities and entrances where pathogenic material such as bacteria or virions may lodge and multiply. The doses of sterilizing radiation afforded by the apparatuses of the present disclosure typically decrease the viral load in these regions.

Higher UV-C length (e.g., 240-280 nm) has significant pathogenic sterilization properties, but in theory may also have deeper penetration to underlying tissue. In certain embodiments, the sterilizing dose of radiation may comprise UV-C higher wavelength light alone or in combination with lower UV-C wavelength light. The spectral distribution may depend upon the target tissue and the pathogen to be sterilized. The spectral distribution may be tuned, for example, by adjusting the number of any individual emitter in the source of sterilizing radiation, or relative positions thereof in the apparatus or device. For example, an apparatus may comprise an increased number lower wavelength UV-C light emitters such that the ratio of lower wavelength UV-C light intensity (e.g., UV-C light having a wavelength of less than 240 nm or less than 230 nm light) as compared to higher wavelength UV-C light (e.g., UV-C light having a wavelength greater than 240 nm or greater than 230 nm) is from 100:1 to 1:1 (e.g., 50:1 to 1:1, 25:1 to 1:1, 10:1 to 1:1, 5:1 to 1:1, 2:1 to 1:1). Similarly, for deeper penetration of the sterilizing dose of radiation, the apparatus may comprise an increased number of higher wavelength UV-C light emitters such that the ratio of lower wavelength UV-C light intensity (e.g., UV-C light having a wavelength of less than 240 nm or less than 230 nm light) as compared to higher wavelength UV-C light (e.g., UV-C light having a wavelength greater than 240 nm or greater than 230 nm) is from 1:1 to 1:100 (e.g., 1:1 to 1:50, 1:1 to 1:25, 1:1 to 1:10, 1:1 to 1:5, 1:1 to 1:2).

In various implementations, the light may be continuously exposed to the material to be sterilized. For example, in certain embodiments, the material to be sterilized may be bombarded with sterilizing radiation for a length of from 0.1 second to 120 minutes (e.g., 0.1 second to 1 second, 1 second to 10 seconds, 10 seconds to 100 seconds, 1 second to 1 minute, 1 minute to 10 minutes, 10 minutes to 30 minutes, 30 minutes to 60 minutes, 60 minutes to 120 minutes). The length of sterilization may be based on the target area of treatment, the pathogen, or combinations thereof. Additionally, the intensity of the sterilizing does may be from 1 mJ/cm² to 1 J/cm² (e.g., 1 mJ/cm² to 100 mJ/cm², 100 mJ/cm² to 1 J/cm²).

In some embodiments, the objects may comprise a sterilizing radiation source capable of emitting a sterilizing dose to the surface of the object; wherein the sterilizing radiation source may be embedded within or otherwise positioned to pass the sterilizing radiation through a transparent or translucent body of the object. In these configurations, the sterilizing radiation may be dispersed and/or pass through the transparent or translucent body of the object such that the light is emitted from the surface to the air at the air/surface interface. The source of the sterilizing radiation may be located within a transparent or translucent material such that the sterilizing radiation is transmitted through the material to the air interface with the surface. In some embodiments, the source of sterilizing radiation is not located within the transparent or translucent material. In some embodiments, the sterilizing radiation is delivered to the transparent or translucent material via an optical wave guide such as optical fibers or transparent dielectric waveguides (e.g., those made of plastic and/or glass).

The transparent or translucent surface is typically composed of material appropriate for the object in terms of mechanical properties such as flexibility, tactile feeling, and biocompatibility. For example, the transparent or translucent surface may be composed of glass, plastic, resin, or combination thereof. Exemplary materials which may be used in objects requiring a higher degree of rigidity such as doorknobs, toilets, toilet flush handles, eyeglasses, shopping cart handles, implements for holding and storing food, or countertops may include polycarbonates, acrylic, modified acrylics, or combinations thereof. For example, the transparent or translucent surface may comprise one or more of a polyester resin such as an aromatic polyester or an aliphatic polyester, an acrylic resin such as polymethyl methacrylate, polyethyl methacrylate, or a vinyl cyclohexane-methyl (meth)acrylate copolymer, a poly(meth)acrylimide resin; a polycarbonate resin such as an aromatic polycarbonate or an aliphatic polycarbonate; a polyolefin resin such as polyethylene, polypropylene, polybutene-1, or poly-4-methyl-pentene-1; a cellulose resin such as cellophane, triacetylcellulose, diacetylcellulose, or acetylcellulose butyrate; a styrene resin such as polystyrene, an acrylonitrile-styrene copolymer (AS resin), an acrylonitrile-butadiene-styrene copolymer resin (ABS resin), a styrene-ethylene-propylene-styrene copolymer, a styrene-ethylene-ethylene-propylene-styrene copolymer, or a styrene-ethylene-butadiene-styrene copolymer; a cyclic hydrocarbon resin such as an ethylene-norbornene copolymer; a polyamide resin such as nylon 6, nylon 66, nylon 12, or nylon 11; a polyvinyl chloride resin such as polyvinyl chloride or a vinyl chloride-vinyl acetate copolymer; a polyvinylidene chloride resin; a fluorine-containing resin such as polyvinylidene fluoride; polyvinyl alcohol, ethylene vinyl alcohol, polyether ether ketone, polyimide, polyurethane, polyetherimide, polysulfone, a polyethersulfone polyarylate resin, and a polymer type urethane acrylate resin. In some embodiments, the transparent or translucent surface may comprise a flexible plastic such as polyethyelene.

Similarly, the thickness of the transparent or translucent surface may vary. For example, the transparent or translucent surface may be more than 1 mm thick or more than 10 mm thick or more than 100 mm thick or more than 1 cm thick, or more than 2 cm thick or more than 3 cm thick or more than 4 cm thick or more than 5 cm thick or more than 6 cm thick or more than 7 cm thick or more than 8 cm thick or more than 10 cm thick or more than 50 cm thick or from 1 mm to 50 cm thick or from 10 mm to 20 cm thick or from 100 mm to 10 cm thick or from 1 cm to 50 cm thick or from 1 cm to 20 cm thick or from 1 cm to 10 cm thick or from 5 cm to 20 cm thick.

The objects and apparatuses of the present disclosure may afford increased exposure time and/or intensity as compared to sterilization techniques involving bombarding the surface with radiation from the air to the surface. Additionally, by providing increased or constant exposure, the pathogenic microorganism (e.g., bacteria, virion) The sterilizing radiation may be any electromagnetic radiation capable of sterilizing the surface and decreasing the pathogenic agents. For example, the sterilizing radiation may be ultraviolet radiation (e.g., UV-C radiation which may have a wavelength of less than 300 nm or a wavelength of from 200 to 300 nm), optical radiation, infrared radiation, or combinations thereof. The power intensity of radiation at the surface may be (which correlates with the sterilizing dose), for example, less than 100 mJ/cm² less than 60 mJ/cm² or less than 50 mJ/cm² or less than 40 mJ/cm² or less than 30 mJ/cm² or less than 20 mJ/cm² or less than 10 mJ/cm² or less than 5 mJ/cm² or from 1 mJ/cm² to 60 mJ/cm² or from 1 mJ/cm² to 25 mJ/cm² or from 1 mJ/cm² to 10 mJ/cm².

In some embodiments, the surface comprises a coating at the air/surface interface. The coating may allow for diffraction of sterilizing radiation at the coating/surface interface resulting from different refractive indexes between the two materials and the sterilizing radiation is provided such that a sterilizing dose of radiation is provided at the air/coating interface. In some embodiments, the surface is uncoated. In various implementations, the surface does not include a coating of a UV active photocatalyst, such as a TiO₂ photocatalyst. In some embodiments, the surface includes a coating of a UV active photocatalyst, such as a TiO₂ photocatalyst.

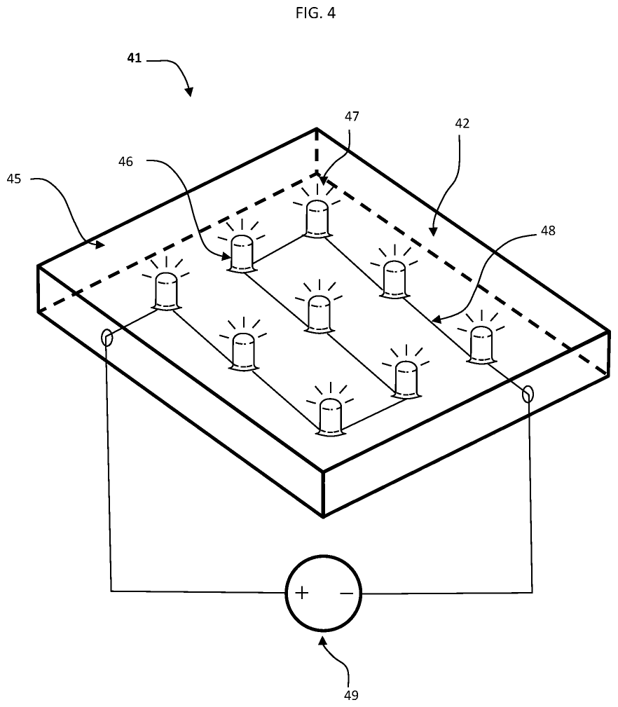

Referring now to FIG. 4, a perspective view of the transparent or translucent material 42 of object 41 is shown. The transparent or translucent material 42 comprises an air interface surface 5. Within the transparent or translucent material 42, are several individual emitters 46 such as light emitting diodes, arc lamps, excimer lamps, or combinations thereof, which emit the light 47 with a sterilizing dose of radiation to the air interface surface 45. In some embodiments, the transparent or translucent material has one or more light emitting diodes (e.g. two or more, three or more, four or more, five or more, ten or more, twenty or more, fifty or more) embedded within the transparent or translucent material. In some embodiments, the light emitting diodes are adjacent to the transparent or translucent material. In the embodiment depicted, each light emitting diode is connected in parallel with the same voltage source 49. In some embodiments, the light emitting diodes are connected in series or parallel with one or more voltage or power sources sufficient to induce emission of a sterilizing dose of radiation.

The transparent or translucent materials may have additives designed to alter the material. For example, the transparent or translucent surface be colored with any colorant. In some embodiments, the colorant does not absorb the sterilizing radiation (e.g., UV-C radiation) or is dispersed in an amount such that the colorant absorbs less than 90% or less than 75% or less than 50% or less than 40% or less than 30% or less than 20% or less than 10% or less than 5% or less than 10% of the sterilizing radiation as compared to an otherwise identical material without the colorant. In various implementations, the transparent or translucent material may comprise one or more light diffusing or light refracting agents in order to refract the sterilizing radiation in the interior of the material. The diffraction may cause the sterilizing radiation to be directed towards the surface/air interface. For example, the transparent or translucent material comprises diffracting or refracting agents (e.g., mica, aluminum) which may diffract or reflect light towards the air interface surface in order to provide the sterilizing dose of radiation to the intended medium.

Other objects may include the transparent or translucent surfaces coupled with sanitizing radiation as described herein. Referring now to FIG. 5, nasal cannula 50 comprises or is composed of a translucent or transparent material between air inputs and outputs 51, 52, and 53. As described herein, the entire surface may have sanitizing radiation passed therethrough in order to sanitize the surface. In the embodiment depicted, nasal cannula 50 comprises an optical waveguide fiber 56 connected to a radiation source and into the surface of the portions of the nasal cannula which are inserted into a user's mouth and/or nose.

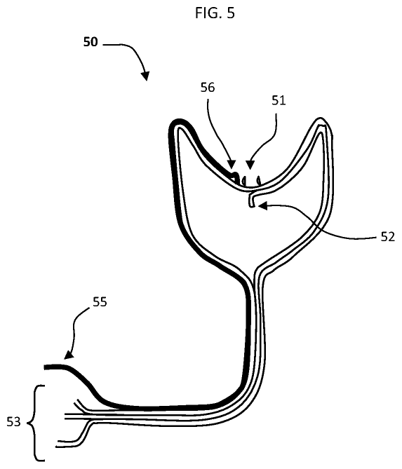

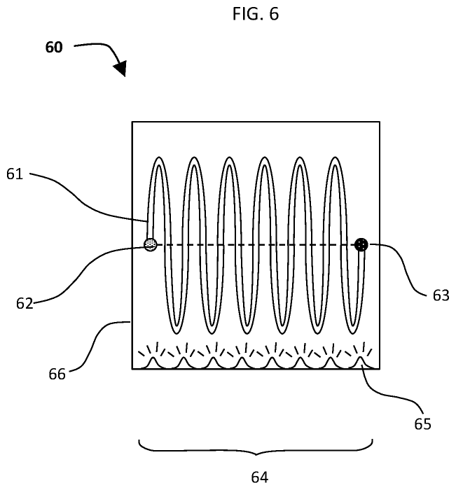

The apparatus may also allow for delivery of sterilized air as required such as delivery of air in a canula or face mask. Referring now to FIG. 6, apparatus 60 comprises:
a) solid enclosure 61 having an internal chamber configured to allow air to pass through the internal chamber (e.g., a tube);
b) an air intake port 62 wherein ambient air is delivered to the internal chamber of solid enclosure 61;
c) an air exhaust port 63 in fluid communication with air intake port 62 via solid enclosure 61; and
d) a sterilizing radiation source 64 comprising one or more individual emitters 65 capable of emitting a sterilizing dose of radiation delivered to the air present in the internal chamber during passage through the apparatus (e.g., the air flowing through the internal chamber from air intake port 62 to air exhaust port 63).
   Each of these components may be contained in housing 66. Such an apparatus may be integrated into air delivery devices, such as cannula, ventilators, or facemasks. For example, the air exhaust port may be connected to the delivery member of a cannula or to the air intake port of a cannula or mask prior to delivery to a subject. In some embodiments, the air intake port 62 may be connected to a source of air such as a pump creating a positive pressure and flow of ambient air to a subject and/or the air exhaust port 63 may be connected to a device which delivers air such as a cannula, ventilator, or facemask. The internal chamber may be dimensioned to allow for increased exposure to the sterilizing radiation produced from sterilizing radiation source 64 by increasing the pathlength of the air in the sterilizing region of the device (e.g., as compared to the distance between the air intake and outtake ports as denoted by the dashed line in FIG. 6). In some embodiments, the implement may have a length of less than 2000 cm or less than 1000 cm or less than 500 cm or less than 300 cm or less than 200 cm or less than 100 cm or less than 50 cm or less than 30 cm. In various implementations, the implement may have a length of from 10 cm to 500 cm or from 30 cm to 400 cm or from 30 cm to 200 cm.

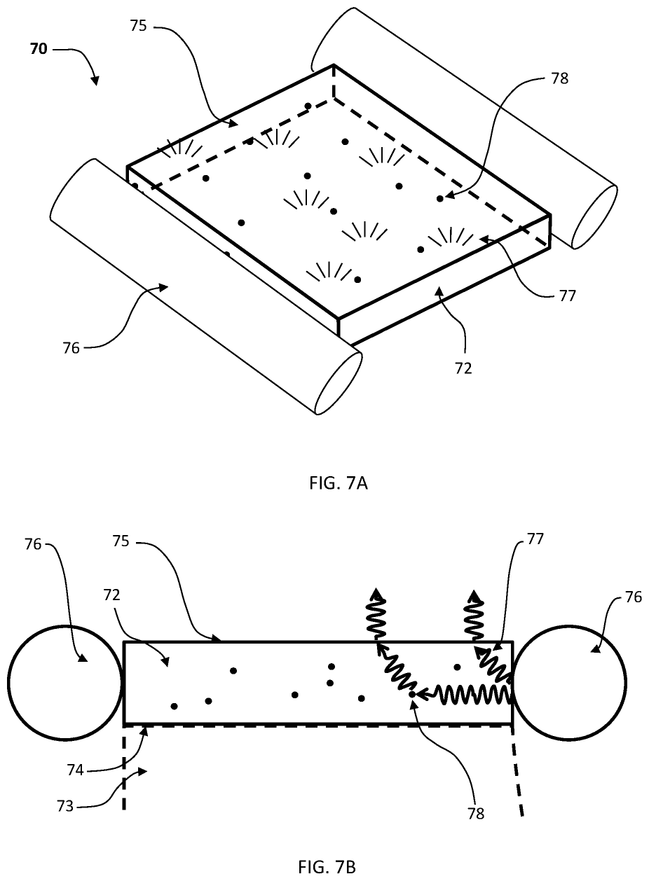

Referring now to FIGS. 7A and 7B, an apparatus having sanitizing radiation at a surface is illustrated. The transparent or translucent surface 72 of object 70 comprises two mercury lamps 76 disposed at opposite ends of translucent surface 72. In some embodiments, the object comprises one or more mercury lamps (e.g., two or more, three or more, four or more, five or more, etc.). As can be seen in FIG. 2B, the transparent or translucent material 72 may be attached to another surface creating a surface to transparent surface interface 74. In some embodiments, surface 73 may comprise a radiation source such as a mercury lamp or one or more light emitting diodes. The transparent or translucent material may be attached, for example, adhesive, or with or connection devices such as snaps, screws, bolts or combinations thereof. In some embodiments, the internal interface 74 may be coated with one or more reflective layers such that sterilizing radiation may reflect of internal interface 74 and be directed to the air interface 75. The light emission sources 76 emit light to pass through the transparent or translucent material.

The transparent or translucent materials may have additives designed to alter the material. For example, the transparent or translucent surface be colored with any colorant. In some embodiments, the colorant does not absorb the sterilizing radiation (e.g., UV-C radiation) or is dispersed in an amount such that the colorant absorbs less than 90% or less than 75% or less than 50% or less than 40% or less than 30% or less than 20% or less than 10% or less than 5% or less than 10% of the sterilizing radiation as compared to an otherwise identical material without the colorant. In various implementations, the transparent or translucent material may comprise one or more light diffusing or light refracting agents in order to refract the sterilizing radiation in the interior of the material. The diffraction may cause the sterilizing radiation to be directed towards the surface/air interface. As can be seen in FIG. 2B, the transparent or translucent material comprises diffracting or refracting agents 78 (e.g., mica, aluminum, etc.) may diffract or reflect light towards the air interface surface 75 in order to provide the sterilizing dose of radiation at the surface as the light passes through the material and to the interface.

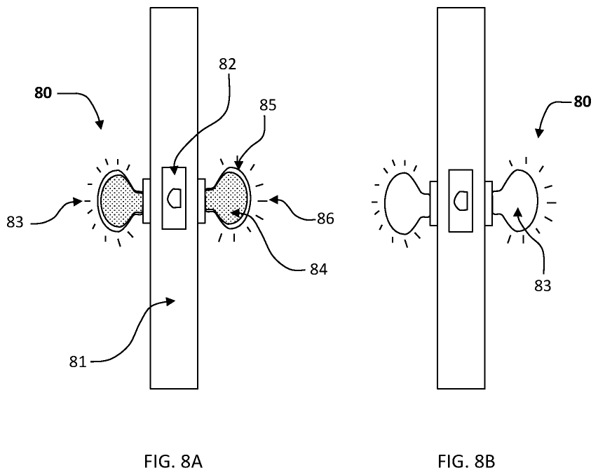

An example of an object utilizing the sterilizing surfaces of the present disclosure is illustrated in FIG. 8A. FIG. 8A shows the cross-sectional view of a door 81 having locking mechanism 82 and a doorknob 80 which emits sterilizing radiation. The door knob comprises first surface 84 with the transparent or translucent surface attached thereto. In some embodiments, the power source or source of radiation is impregnated within the first surface. Surrounding a portion of the first material 84 is the transparent or translucent material 85 capable of emitting light 86 through its air interface. As can be seen, light 83 and 86 is emit from each doorknob on door 81. FIG. 8B shows a doorknob 80 entirely composed of the translucent or transparent material 83. In some embodiments, translucent or transparent material 83 may have light delivered to it (e.g., via waveguides, with radiation sources proximal thereto) and/or have one or more radiation sources impregnated within the transparent or translucent material.

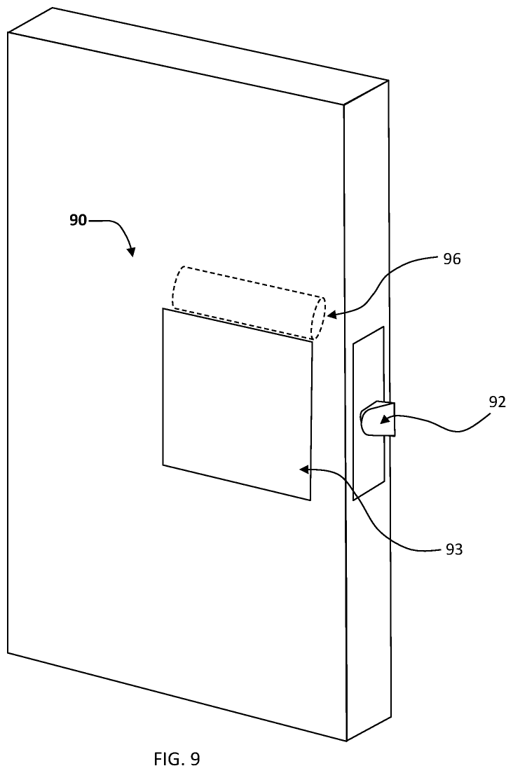

Referring now to FIG. 9, a perspective view of another door opening device 90 is illustrated. Door opening device 90 comprises a panel 93 comprising the transparent or translucent surface. In some embodiments, the exposed surface of the panel may be asymmetric, triangular, rectangular, square, circular, ovoid, rhomboid, parallelepiped, pentagonal, hexagonal, septagonal, octagonal, or nonagonal. In some embodiments the sanitizing dose of radiation is emit over a surface area of rom 1 cm² to 1000 cm² (e.g., from 1 10 cm² to 100 cm²). A radiation source (e.g., a mercury lamp) 46 is also embedded in the door proximal to the panel (e.g., the radiation source is within 5 cm or within 4 cm or within 3 cm or within 2 cm or within 1 cm of the edge of the transparent or translucent material) such that a sanitizing dose of radiation may be emitted from the air interface of panel 93. Panel 93 may be connected to locking mechanism 93 such that when a user presses on the surface of panel 93, the locking mechanism disengages allowing for the door to be pushed in open. In some embodiments, the door does not comprise a locking mechanism.

### SPECIFIC EMBODIMENTS

Non-limiting specific embodiments are described below each of which is considered to be within the present disclosure.

Specific Embodiment 1. An apparatus for delivering sterilized air to a subject during inhalation comprising:
a) a solid enclosure having an internal chamber configured to allow air to pass through the internal chamber and be delivered to the subject;
b) a sterilizing radiation source capable of emitting:
   i) a sterilizing dose of radiation delivered to the air present in the internal chamber prior to delivery to the subject;
   ii) a sterilizing dose of radiation delivered to one or more of the oral cavity of the subject, the nasal cavity of the subject, or
   iii) any combination of two or more of the foregoing.

Specific Embodiment 2. The apparatus according to Specific Embodiment 1, wherein said sterilizing radiation source emits a sterilizing dose of radiation comprising UV-C light.

Specific Embodiment 3. The apparatus according to Specific Embodiment 1, wherein said sterilizing radiation source emits photons having one or more wavelengths from 10 nm to 400 nm.

Specific Embodiment 4. The apparatus according to Specific Embodiment 1, wherein said sterilizing radiation source emits radiation to be delivered to the mucosa of the subject, and said sterilizing radiation source emits radiation comprising two spectral maximums having different penetration depths in the mucosa.

Specific Embodiment 5. The apparatus according to Specific Embodiment 4, wherein the sterilizing radiation having one of said spectral maximums is surface sterilizing radiation which predominantly sterilizes the surface of the mucosa.

Specific Embodiment 6. The apparatus according to Specific Embodiment 5, wherein said surface sterilizing radiation has a spectral maximum of less than 240 nm.

Specific Embodiment 7. The apparatus according to Specific Embodiment 4, wherein the sterilizing radiation having one of said spectral maximums is penetrating sterilizing radiation which predominantly sterilizes the mucosa beneath its surface.

Specific Embodiment 8. The apparatus according to Specific Embodiment 7, wherein said surface sterilizing radiation has a spectral maximum of more than 240 nm.

Specific Embodiment 9. The apparatus according to Specific Embodiment 1, wherein said sterilizing radiation source emits photons having one or more local spectral maximum wavelengths selected from 108 nm, 126 nm, 146 nm, 158 nm, 165 nm, 172 nm, 175 nm, 190 nm, 193 nm, 207 nm, 210 nm, 215 nm, 222 nm, 23 5 nm, 248 nm, 253 nm, 259 nm, 282 nm, 289 nm, 308 nm, 342 nm, 351 nm, or 365 nm.

Specific Embodiment 10. The apparatus according to Specific Embodiment 1, wherein said sterilizing radiation source emits photons having a spectral maximum wavelength greater than 240 nm and said sterilizing radiation source emits photons having a spectral maximum wavelength less than 240 nm.

Specific Embodiment 11. The apparatus according to Specific Embodiment 1, wherein said sterilizing radiation source comprises a light emitting diode (LED), a mercury lamp, or an excimer lamp, individually or in combinations of two or more thereof.

Specific Embodiment 12. The apparatus according to Specific Embodiment 1, wherein said apparatus is a face mask;

the solid enclosure is dimensioned to be fit around the mouth and the nose of the subject during inhalation and a portion of the internal cavity is formed between the face and the solid enclosure;
the solid enclosure is dimensioned to be positioned on the chest of the subject, the shoulders of the subject, or the chest and the shoulders of the subject during inhalation; and
the face mask is supported by the positioning on the chest of the subject, the shoulders of the subject, or the chest and the shoulders of the subject during inhalation.

Specific Embodiment 13. The apparatus according to Specific Embodiment 12, wherein said solid enclosure comprises an air intake port and a tube comprising a distal end having an air delivery port dimensioned to be inserted into the nose of the subject and a distal end and air may flow through the distal end;

wherein said air intake port is in fluid communication with the distal and ambient air may flow through the air intake port, through the tube, and out the air delivery port;
said tube is transparent or translucent to the sterilizing radiation; and
said sterilizing dose of radiation is delivered to the ambient air flowing through the tube.

Specific Embodiment 14. The apparatus according to Specific Embodiment 13, wherein said tube length is longer than the distance between the air intake port and the user's nose.

Specific Embodiment 15. The apparatus according to Specific Embodiment 12, further comprising one or more voice modulators.

Specific Embodiment 16. The apparatus according to Specific Embodiment 1, wherein said apparatus is a nasal cannula or catheter.

Specific Embodiment 17. The apparatus according to Specific Embodiment 1, wherein said apparatus, one or more air intake ports, one or more air outtake ports, individually or in combination thereof; wherein said air intake ports are configured to regulate the flow of air into the internal cavity, out of the internal cavity, or into and out of the internal cavity.

Specific Embodiment 18. The apparatus according to Specific Embodiment 1, further comprising one or more delivery ports wherein air passes through the port for delivery to the subject for inhalation.

Specific Embodiment 19. The apparatus according to Specific Embodiment 1, comprising:
a) a solid enclosure having an internal chamber configured to allow air to pass through the internal chamber;
b) a sterilizing radiation source capable of emitting a sterilizing dose of radiation delivered to the air present in the internal chamber during passage through the apparatus;
c) an air intake port wherein ambient air is delivered to the internal chamber of solid enclosure; and
d) an air exhaust port in fluid communication with air intake port via solid enclosure.

Specific Embodiment 20. An apparatus for sterilizing the nasal cavity of a subject comprising:
a) one or members dimensioned to be inserted into the nasal cavity; and
b) a sterilizing radiation source capable of emitting a sterilizing dose of radiation to the nasal cavity when the member is inserted in the nasal cavity.

Specific Embodiment 21. The apparatus according to Specific Embodiment 20, wherein said sterilizing radiation source emits a sterilizing dose of radiation comprising UV-C light.

Specific Embodiment 22. The apparatus according to Specific Embodiment 20, wherein said sterilizing radiation source emits photons having one or more wavelengths from 10 nm to 400 nm.

Specific Embodiment 23. The apparatus according to Specific Embodiment 20, wherein said sterilizing radiation source emits radiation to be delivered to the mucosa of the subject, and

said sterilizing radiation source emits radiation comprising two spectral maximums having different penetration depths in the mucosa.

Specific Embodiment 24. The apparatus according to Specific Embodiment 23, wherein the sterilizing radiation having one of said spectral maximums is surface sterilizing radiation which predominantly sterilizes the surface of the mucosa.

Specific Embodiment 25. The apparatus according to Specific Embodiment 24, wherein said surface sterilizing radiation has a spectral maximum of less than 240 nm.

Specific Embodiment 26. The apparatus according to Specific Embodiment 23, wherein the sterilizing radiation having one of said spectral maximums is penetrating sterilizing radiation which predominantly sterilizes the mucosa beneath its surface.

Specific Embodiment 27. The apparatus according to Specific Embodiment 26, wherein said surface sterilizing radiation has a spectral maximum of more than 240 nm.

Specific Embodiment 28. The apparatus according to Specific Embodiment 20, wherein said sterilizing radiation source emits photons having one or more spectral maximum wavelengths selected from 108 nm, 126 nm, 146 nm, 158 nm, 165 nm, 172 nm, 175 nm, 190 nm, 193 nm, 207 nm, 222 nm, 248 nm, 253 nm, 259 nm, 282 nm, 289 nm, 308 nm, 342 nm, and 351 nm.

Specific Embodiment 29. The apparatus according to Specific Embodiment 20, wherein said sterilizing radiation source emits photons having a spectral maximum wavelength greater than 240 nm and said sterilizing radiation source emits photons having a spectral maximum wavelength less than 240 nm.

Specific Embodiment 30. The apparatus according to Specific Embodiment 20, wherein said sterilizing radiation source comprises a light emitting diode (LED), a mercury lamp, or an excimer lamp, individually or in combinations of two or more thereof.

Specific Embodiment 31. An object comprising a surface composed of a transparent or translucent material positioned at the air interface of the object and a sterilizing radiation source capable of emitting a sterilizing dose at the interface after transmission of the radiation through to the air interface.

Specific Embodiment 32. The object according to Specific Embodiment 31, wherein the radiation source is embedded within the transparent or translucent material.

Specific Embodiment 33. The object according to Specific Embodiment 31, wherein the radiation source is proximal to the transparent or translucent material.

Specific Embodiment 34. The object according to Specific Embodiment 31, wherein the radiation is delivered to the transparent or translucent material via an optical wave guide.

Specific Embodiment 35. The object according to Specific Embodiment 31, wherein the object is a doorknob, door handle, toilet, flush handle of a toilet, hair net, shopping cart handle, implement for holding, transferring or storing food, facemask. or a flat surface on the door or counter top.

Specific Embodiment 36. The object according to Specific Embodiment 31, wherein the object is eye glasses, face shield, face mask, nasal cannula, nose ring or stud, arm band, wound cover, or bandage.

Specific Embodiment 37. The object according to Specific Embodiment 31, wherein the object is a medical implant.

Specific Embodiment 38. The object according to Specific Embodiment 37, wherein the medical implant is in the form of a capsule, bulb, catheter, or electrode.

As various changes can be made in the above-described subject matter without departing from the scope and spirit of the present disclosure, it is intended that all subject matter contained in the above description, or defined in the appended claims, be interpreted as descriptive and illustrative of the present disclosure. Many modifications and variations of the present disclosure are possible in light of the above teachings. Accordingly, the present description is intended to embrace all such alternatives, modifications and variances which fall within the scope of the appended claims.

All documents cited or referenced herein and all documents cited or referenced in the herein cited documents, together with any manufacturer's instructions, descriptions, product specifications, and product sheets for any products mentioned herein or in any document incorporated by reference herein, are hereby incorporated by reference, and may be employed in the practice of the disclosure.

1. An apparatus for delivering sterilized air to a subject during inhalation comprising:
a) a solid enclosure having an internal chamber configured to allow air to pass through the internal chamber and be delivered to the subject;
b) a sterilizing radiation source capable of emitting:
i) a sterilizing dose of radiation delivered to the air present in the internal chamber prior to delivery to the subject;
ii) a sterilizing dose of radiation delivered to one or more of the oral cavity of the subject, the nasal cavity of the subject, or
iii) any combination of two or more of the foregoing.

2. The apparatus according to claim 1, wherein said sterilizing radiation source emits a sterilizing dose of radiation comprising UV-C light.

3. The apparatus according to claim 1, wherein said sterilizing radiation source emits radiation to be delivered to the mucosa of the subject, and
said sterilizing radiation source emits radiation comprising two spectral maximums having different penetration depths in the mucosa.

4. The apparatus according to claim 3, wherein the sterilizing radiation having one of said spectral maximums is surface sterilizing radiation which predominantly sterilizes the surface of the mucosa.

5. The apparatus according to claim 4, wherein said surface sterilizing radiation has a spectral maximum of less than 240 nm.

6. The apparatus according to claim 1, wherein said sterilizing radiation source emits photons having one or more local spectral maximum wavelengths selected from 108 nm, 126 nm, 146 nm, 158 nm, 165 nm, 172 nm, 175 nm, 190 nm, 193 nm, 207 nm, 210 nm, 215 nm, 222 nm, 23 5 nm, 248 nm, 253 nm, 259 nm, 282 nm, 289 nm, 308 nm, 342 nm, 351 nm, or 365 nm.

7. The apparatus according to claim 1, wherein said sterilizing radiation source emits photons having a spectral maximum wavelength greater than 240 nm and said sterilizing radiation source emits photons having a spectral maximum wavelength less than 240 nm.

8. The apparatus according to claim 1, wherein said sterilizing radiation source comprises a light emitting diode (LED), a mercury lamp, or an excimer lamp, individually or in combinations of two or more thereof.

9. The apparatus according to claim 1, wherein said apparatus is a face mask;
the solid enclosure is dimensioned to be fit around the mouth and the nose of the subject during inhalation and a portion of the internal cavity is formed between the face and the solid enclosure;
the solid enclosure is dimensioned to be positioned on the chest of the subject, the shoulders of the subject, or the chest and the shoulders of the subject during inhalation; and
the face mask is supported by the positioning on the chest of the subject, the shoulders of the subject, or the chest and the shoulders of the subject during inhalation.

10. The apparatus according to claim 9, wherein said solid enclosure comprises an air intake port and a tube comprising a distal end having an air delivery port dimensioned to be inserted into the nose of the subject and a distal end and air may flow through the distal end;
wherein said air intake port is in fluid communication with the distal and ambient air may flow through the air intake port, through the tube, and out the air delivery port;
said tube is transparent or translucent to the sterilizing radiation; and
said sterilizing dose of radiation is delivered to the ambient air flowing through the tube.

11. The apparatus according to claim 1, further comprising one or more delivery ports wherein air passes through the port for delivery to the subject for inhalation.

12. The apparatus according to claim 1, comprising:
a) a solid enclosure having an internal chamber configured to allow air to pass through the internal chamber;
b) a sterilizing radiation source capable of emitting a sterilizing dose of radiation delivered to the air present in the internal chamber during passage through the apparatus;
c) an air intake port wherein ambient air is delivered to the internal chamber of solid enclosure; and
d) an air exhaust port in fluid communication with air intake port via solid enclosure.

13. An apparatus for sterilizing the nasal cavity of a subject comprising:
a) one or members dimensioned to be inserted into the nasal cavity; and
b) a sterilizing radiation source capable of emitting a sterilizing dose of radiation to the nasal cavity when the member is inserted in the nasal cavity.

14. The apparatus according to claim 13, wherein said sterilizing radiation source emits a sterilizing dose of radiation comprising UV-C light.

15. The apparatus according to claim 13, wherein said sterilizing radiation source emits photons having one or more spectral maximum wavelengths selected from 108 nm, 126 nm, 146 nm, 158 nm, 165 nm, 172 nm, 175 nm, 190 nm, 193 nm, 207 nm, 222 nm, 248 nm, 253 nm, 259 nm, 282 nm, 289 nm, 308 nm, 342 nm, and 351 nm.

16. The apparatus according to claim 13, wherein said sterilizing radiation source emits photons having a spectral maximum wavelength greater than 240 nm and said sterilizing radiation source emits photons having a spectral maximum wavelength less than 240 nm.

17. The apparatus according to claim 13, wherein said sterilizing radiation source comprises a light emitting diode (LED), a mercury lamp, or an excimer lamp, individually or in combinations of two or more thereof.

18. An object comprising a surface composed of a transparent or translucent material positioned at the air interface of the object and a sterilizing radiation source capable of emitting a sterilizing dose at the interface after transmission of the radiation through to the air interface.

19. The object according to claim 18, wherein the radiation source is embedded within the transparent or translucent material.

20. The object according to claim 18, wherein the object is a doorknob, door handle, toilet, flush handle of a toilet, hair net, shopping cart handle, implement for holding, transferring or storing food, facemask. or a flat surface on the door or counter top.
